SEQUENCE LISTING (3 sequences)

SEQ ID NO: 1 - DNA, 23 bp, Artificial Sequence
  DNA oligonucleotide with 6-FAM as fluorescence dye at its 5′ end and BHQ1 as quencher at its 3′ end
ggaggcgatctatgtcttgtcca

SEQ ID NO: 2 - DNA, 24 bp, Artificial Sequence
  Oligonucleotide primer sequence
acatcttaccgcaactacgaccat

SEQ ID NO: 3 - DNA, 24 bp, Artificial Sequence
  Oligonucleotide primer sequence
tagcatagtctttgtcccaccgta

(19) United States
(12) Patent Application Publication
(10) Pub. No.: US 2013/0022984 A1
(43) Pub. Date: Jan. 24, 2013

(54) METHOD FOR THE NON-SPECIFIC ENRICHMENT OF MICROORGANISMS

(71) Applicant: Frank Narz, Hilden (DE)

(72) Inventor: Frank Narz, Hilden (DE)

(73) Assignee: Qiagen GmbH, Hilden (DE)

(21) Appl. No.: 13/635,645

(22) PCT Filed: Apr. 15, 2011
(86) PCT No.: PCT/EP2011/055972

§ 371 Date: Sep. 17, 2012

(30) Foreign Application Priority Data
Apr. 15, 2010 (EP) 10003967.6

(51) Int. Cl. G01N 33/569; G01N 21/76; C12Q 1/68

(52) U.S. Cl. 435/6.12; 435/7.21

(57) ABSTRACT

The present invention relates to a method for the non-specific enrichment of microorganisms from complex starting materials, wherein the starting materials containing the microorganisms are brought in contact with cells of the innate immune system, the microorganisms are bound to the cells of the innate immune system and the binding complex is separated from the complex starting material.

The present invention relates to a method for nonspecifically accumulating microorganisms from complex starting materials, wherein the starting materials containing the microorganisms are contacted with cells of the innate immune system, the microorganisms are bound to the cells of the innate immune system, and the binding complex is removed from the complex starting material.

Microorganisms are generally present in a very low concentration in complex starting materials such as, for example, foodstuffs or soil samples. In order to be able to obtain a sufficient number of microorganisms for detection thereof from said starting materials, it is necessary to process relatively large amounts of starting material. Since it is not possible in most methods to process large amounts of starting material with the microorganisms contained therein, it is necessary to accumulate the microorganisms beforehand from said starting materials. This accumulation is complicated by the fact that starting materials such as foodstuffs or soil samples are generally a very complex mixture in which many different organic molecules from different classes of substance with varying physical and chemical properties are present.

For this reason, it is difficult to accumulate microorganisms from said starting materials without co-accumulating further constituents of the starting materials, which might interfere with or inhibit the subsequent treatment of the microorganisms.

Various methods are known from the prior art which make it possible for microorganisms to be accumulated from complex starting materials.

The chemical methods include, for example, the adsorption of microorganisms to ion-exchange matrices. Since most microorganisms have a negative surface charge when the pH is above 5, many microorganisms can bind to anion-exchange matrices. However, on the other hand, since many substances present in the complex starting materials also have a negative surface charge, they also bind to the anion-exchange matrix, greatly limiting the binding capacity of the matrix for microorganisms, with the result that in extreme cases, no microorganisms can bind to the matrix, since all binding sites are already saturated by other constituents of the starting materials.

A further chemical method is the binding of microorganisms to lectins. Lectins are carbohydrate-binding proteins which selectively recognize and bind the surface components of bacteria. The disadvantage of this method is that the lectins preferentially bind to Gram-positive bacteria. Although Gram-negative bacteria are also bound, though with lower affinity, other microorganisms such as, for example, fungi or viruses cannot be bound by the lectins. Consequently, the binding of microorganisms to lectins is restricted to bacteria, more particularly Gram-positive bacteria.

A further chemical method is the separation of microorganisms by aqueous two-phase systems. In this method, the microorganisms are separated from the remaining constituents of the starting material between two fluid phases which are not miscible with one another and which differ from one another in their molecular weight. The disadvantage of this method is that very many constituents of the starting material accumulate in the same phase as the microorganisms, i.e., the microorganisms cannot be removed from all the constituents of the starting material. In addition, the separation into the individual phases is frequently incomplete and leads to loss of some of the microorganisms in the second phase.

The physical methods which have been described for accumulation of microorganisms include, for example, centrifugation. If centrifugation is carried out at a relatively low g-force, this leads to sedimentation of the majority of the constituents of the starting material, whereas the microorganisms remain in the supernatant. However, the microorganisms associated with heavier constituents of the starting material also undergo sedimentation and are therefore lost. On the other hand, by centrifuging at a low g-force, constituents of the starting material which are lighter than the microorganisms cannot be removed from the microorganisms, and so only crude accumulation of microorganisms can be achieved by this method.

A further physical method for accumulating microorganisms is filtration through suitable filters. Filtration has been found to be a relatively unsuitable method, since relatively large constituents of the starting material quickly clog the filter, bringing separation very quickly to an end. In addition, many constituents of the starting material, which may complicate the detection of the microorganisms, are also concentrated together with the microorganisms.

A further method for accumulating microorganisms utilizes the immunoaffinity of microorganisms. In this method, antibodies which can specifically bind a particular species of microorganism are immobilized on a solid phase. Although this method is highly specific, and no further constituents of the starting material bind to the antibodies, this method is not a generic method, since only one particular species or one particular group of microorganisms can bind to the antibodies, whereas other microorganisms present in the sample cannot bind to the antibodies. In addition, this method is fairly costly owing to the need to provide a large number of antibodies.

It is an object of the present invention to overcome the disadvantages of the methods known from the prior art and to provide a generic method for accumulating microorganisms from complex starting materials, in which the microorganisms are present in a higher concentration after removal from the complex starting material.

This object is achieved by a method for nonspecifically accumulating microorganisms from complex starting materials, comprising the following method steps:
a) contacting the microorganism-containing complex starting material with cells of the innate immune system;
b) forming a binding complex between the microorganisms and cells of the innate immune system;
c) removing the binding complex from step b) from the complex starting material.

The method according to the invention is suitable for nonspecifically accumulating microorganisms from a very wide variety of different complex starting materials.

In this connection, accumulation is understood to mean that the microorganisms, after removal of the binding complex from the complex starting material, are present in a higher concentration than in the complex starting material, and this can be achieved, for example, by a reduction of the volume in which the microorganisms are present after the method.

A microorganism is understood to mean all organisms which are recognized as being foreign by the innate immune system of higher organisms. These include, for example, not only Gram-positive and Gram-negative bacteria and persistent states thereof but also fungi and persistent states thereof, viruses and archaebacteria and constituents of these organisms. For the method according to the invention, it is not important whether these microorganisms exhibit metabolic activity or are dead or whether constituents of these microorganisms are involved, so long as they can be recognized as being foreign by the innate immune system.

In a preferred embodiment, the microorganisms to be accumulated are Gram-positive and/or Gram-negative bacteria.

A suitable complex starting material from which the microorganisms are to be accumulated is, in principle, any starting material in which microorganisms are or may be present. These starting materials include, for example, foodstuffs which are to be tested for absence of microorganisms. These include, for example, fruit juices, milk, meat, fruit, sausage products, cheese and further milk products. However, other complex starting materials from which microorganisms are to be detected can also be used as starting materials in the method according to the invention. These include, for example, soil samples or sludge samples or else blood and other body fluids and also cosmetics.

Depending on how the complex starting materials are supplied, it may be necessary to subject the starting materials to a pretreatment before they are contacted with cells of the innate immune system in step a) of the method according to the invention.

For example, it is possible to initially centrifuge the starting materials under conditions in which the microorganisms remain in solution, while constituents of the starting materials pellet under said conditions. In this case, further processing is carried out with the supernatant containing the microorganisms in solution.

However, it is also possible to pellet the microorganisms, while constituents of the starting materials remain in solution under these conditions. In this case, further processing is carried out with the pellet with the microorganisms located therein.

In the case of complex starting materials which are mainly solid, such as soil samples or meat for example, it may be necessary initially for the starting material to be comminuted, homogenized and/or admixed with a liquid before it is contacted with cells of the innate immune system in step a) of the method according to the invention. Means for comminuting and/or homogenizing starting materials are familiar to a person skilled in the art.

Even if the starting materials have initially been comminuted, homogenized and/or admixed with a liquid, it may be necessary for the starting materials to receive yet further pretreatment, for example centrifugation, before they are contacted with the cells of the innate immune system.

Further methods and means with regard to how the complex starting materials can be pretreated are familiar to a person skilled in the art.

In step a) of the method according to the invention, the complex starting material containing the microorganisms is contacted with cells of the innate immune system.

In this connection, the cells of the innate immune system can originate from any desired organism which has an innate immune system capable of recognizing microorganisms as being foreign to the organism. Virtually all multicellular organisms are capable of recognizing microorganisms as being foreign and have an innate immune system. These organisms having cells of an innate immune system include, for example, humans, further mammals and further vertebrates, plants, insects and further invertebrates.

In a preferred embodiment, the cells of the innate immune system originate from mammals.

In a particularly preferred embodiment, the cells of the innate immune system are of human origin.

The cells of the innate immune system can be present isolated as individual cells, or else they can form a cell mass or tissue mass made up of cells of the innate immune system. Moreover, it is possible for the cells of the innate immune system to be located in cell masses or tissue masses in which further cells are to be found which are not part of the innate immune system.

In one embodiment, the cells of the innate immune system are primary cells which have been taken from an organism as individual cells, as a cell mass or as a tissue mass. Said primary cells can be introduced directly into the method according to the invention, or else they can have been cultured further in vitro before their use in the method according to the invention. In a preferred embodiment, the primary cells of the innate immune system are myeloid cells. These include, for example, monocytes, macrophages, neutrophilic, eosinophilic and basophilic granulocytes and natural killer cells. They can be used as a mixture of individual cell types, or else just one cell type can be used.

In a particularly preferred embodiment, the myeloid cells are macrophages.

In a further embodiment, the cells of the innate immune system are immortalized cells of one or more appropriate cell lines which are kept in cell culture.

In a preferred embodiment, said immortalized cells are derived from the circulatory system, such as, for example, leukemia cells, lymphoma cells or myeloma cells.

In a particularly preferred embodiment, the immortalized cells are cells of the cell line THP 1, U937, K562 or a mixture thereof.

Further useful cell lines are familiar to a person skilled in the art.

In step b) of the method according to the invention, the microorganisms bind to the cells of the innate immune system and thus form a binding complex. For the purposes of the present invention, binding is understood to mean any interaction between the microorganism and the cell of the innate immune system. The cells of the innate immune system are capable of recognizing exogenous cells, cellular constituents and organisms at the molecular level and of entering into an interaction with them. In this connection, the cells of the innate immune system do not specifically recognize a particular exogenous microorganism or a part thereof, as is the case, for example, in antibody-antigen interactions. On the contrary, this interaction is nonspecific, and so the cells of the innate immune system can recognize a broad spectrum of different microorganisms or parts of said microorganisms at the molecular level.

The interaction between cell and microorganism can, for example, consist of binding between cell and microorganism in the narrower sense, involving a molecular interaction between these two partners, or else the interaction can consist in the cell of the innate immune system phagocytizing the microorganism or incorporating it into the cell interior in another way. Further means of interaction between cell and microorganism are familiar to a person skilled in the art.

In one embodiment, the cells of the innate immune system exhibit metabolic activity. In this connection, the cells are capable of carrying out some or most of their metabolic pathways and/or biological processes. These include, for example, the interaction with other cells, cell masses or tissues or else the phagocytosis of substances and microorganisms recognized as being exogenous. The capacity for cell division is also included, so long as the cells are capable of it owing to their degree of differentiation.

In a further embodiment, the cells of the innate immune system no longer exhibit metabolic activity. Said cells can, for example, be fixed and are as a result protected from autolysis, and so their cellular structures are largely preserved. The fixing of cells is achieved, for example, by the crosslinking of cellular constituents by means of aldehydes such as formaldehyde or glutaraldehyde. A further method for fixation is, for example, fixation by means of dehydration, as can be achieved by chemicals such as acetone or alcohols such as, for example, ethanol or methanol. Further methods for fixing cells to largely preserve the cellular structures are familiar to a person skilled in the art.

Irrespective of whether the cells still exhibit metabolic activity or not, they can be immobilized on a solid phase. The immobilization can be achieved via covalent bonding or via other mechanisms, such as van der Waals forces, binding via functional groups, receptor-ligand binding, antibody-antigen binding or electrostatic interaction. Further means of immobilizing cells to solid phases are familiar to a person skilled in the art.

Suitable solid phases are, in principle, all possible shapes and materials. Examples of solid phases are planar, convex or concave surfaces, magnetic and nonmagnetic particles, coatings of reaction vessels, microtiter plates, microscope slides and many more. The solid phases can be produced from any desired material, so long as it is possible for cells of the innate immune system to be directly or indirectly immobilized on said solid phases. Many other suitable solid phases which can be used for the method according to the invention are familiar to a person skilled in the art.

Irrespective of whether the cells of the innate immune system are immobilized on a solid phase or not, said cells comprise intracellularly magnetic particles in a particular embodiment. The magnetic particles can be ferromagnetic, ferrimagnetic or superparamagnetic particles. Many different methods are familiar to a person skilled in the art with regard to how such magnetic particles can reach the cell interior. Possibilities are, for example, incorporation by phagocytosis, importation by means of various transfection methods, or bombardment of the cells using a particle gun.

In step c) of the method according to the invention, the binding complex from step b) is removed from the complex starting material.

Means of separating the binding complex from the complex starting material are sufficiently known from the prior art. If the density of the binding complex is different to that of the complex starting material, the binding complex can be simply removed from the complex starting material by centrifugation. A further means of removal is filtration, if the size of the binding complex is different to that of the complex starting material. A further means of removal consists in using antibodies which are directed against an epitope on the surface of the cells of the innate immune system.

The removal of the complex starting material is facilitated if the cells of the innate immune system are coupled to a solid phase, since the solid phase which now has situated on it the complex of cells of the innate immune system with the microorganisms can be easily removed from the remaining constituents of the complex starting material. Removal can, for example, be achieved by simple withdrawal of the solid phase from the binding reaction containing the complex starting material or else, for example, by decanting the binding reaction, in which case the binding complex of cells of the innate immune system with the microorganisms bound thereto is retained in the reaction vessel.

If the solid phase is magnetic, or if the cells of the innate immune system have incorporated magnetic particles, the separation of the binding complex from the remaining constituents of the binding reaction containing the complex starting material can be easily achieved by magnetic separation.

Further means of separating the binding complex from the binding reaction are familiar to a person skilled in the art.

After the binding complex has been removed from the complex starting material in step c), the binding complex of cells of the innate immune system with microorganisms can be washed if necessary so that remnants of the complex starting material which may still be present are eliminated from the binding complex.

After the microorganisms have been nonspecifically accumulated by the method according to the invention, said microorganisms can then be detected and/or quantified in a specific manner using methods familiar to a person skilled in the art.

For example, the microorganisms can be detected by means of particular proteins on their surface. The detection of said proteins can be achieved, for example, by means of immunofluorescence, Western blot, FACS, flow cytometry and further protein detection methods known to a person skilled in the art.

Alternatively, the microorganisms can also be detected and quantified by means of their nucleic acids. Means for this are, for example, nucleic acid hybridization techniques, which include, for example, Northern blots, Southern blots, microarrays and dot blots.

A further means of specifically detecting microorganisms via their nucleic acid sequence consists in amplification techniques such as PCR with their possible variants such as, for example, end-point PCR or real-time PCR and in isothermal amplification methods such as, for example, rolling circle amplification (RCA) with their possible variants.

Further means of detecting microorganisms via their nucleic acids are familiar to a person skilled in the art.

### FIGURES

FIG. 1:

Analysis of the binding of various fluorescently labeled microorganisms of varying number to THP1 cells by means of flow cytometry. The filled area represents the zero control, the solid line 10⁶ microorganisms, the dashed line 10⁷ microorganisms, the dotted line 10⁸ microorganisms.

FIG. 2:

Analysis of the binding of fluorescently labeled Bacillus subtilis of varying number to THP1, U937 or K562 cells by means of flow cytometry. The filled area represents the zero control, the solid line 10⁵ microorganisms, the dashed line 10⁷ microorganisms.

FIG. 3:

Analysis of the binding of fluorescently labeled Bacillus subtilis of varying number to fixed THP1 cells by means of flow cytometry. The filled area represents the zero control, the solid line 10⁵ microorganisms, the dashed line 10⁷ microorganisms.

FIG. 4:

Analysis of the binding of fluorescently labeled Bacillus subtilis of varying number to THP1 cells in milk at different times by means of flow cytometry. The filled area represents the zero control, the solid line 10⁶ microorganisms, the dashed line 10⁸ microorganisms.

FIG. 5:

Detection of THP1-bound bacteria by means of RT-PCR. The X-axis indicates the varying number of the bacteria used and also the manner in which the binding complex was removed from the starting material. The Y-axis indicates the cT value.

### EXAMPLES

The intention of the following examples is to further elucidate the invention, without limiting the invention to the exemplary embodiments.

The following oligonucleotides or primer/probe sequences were used:

BacSub-Probe:
5′-6-FAM-GGAGGCGATCTATGTCTTGTCCA-BHQ1
BacSub-FWD:
5′-ACATCTTACCGCAACTACGACCAT
BacSub-REV:
5′-TAGCATAGTCTTTGTCCCACCGTA

### Example 1

### Binding of Various Bacteria to THP1 Cells

Escherichia coli (Gram-negative bacterium), Bacillus subtilis and Corynebacterium glutamicum (both Gram-positive bacteria) were added (10⁹ in each case) to 1 ml of PBS and stained with the fluorescent dye SYTO BC from the Bacteria Counting Kit (Invitrogen, Carlsbad, USA) in the dark with rotation for 10 min at room temperature. Subsequently, the bacteria were centrifuged down at 14 000 rpm for 5 min and washed with 1 ml of PBS. 10⁶, 10⁷ or 10⁸ of these fluorescently labeled bacteria were added in each case to 2×10⁵ cells of the human monocyte cell line THP1 and incubated with agitation for 30 min at 37° C. After centrifugation at 1000 rpm for 5 min, the binding complex was washed with PBS and, after recentrifugation, analyzed in a flow cytometer.

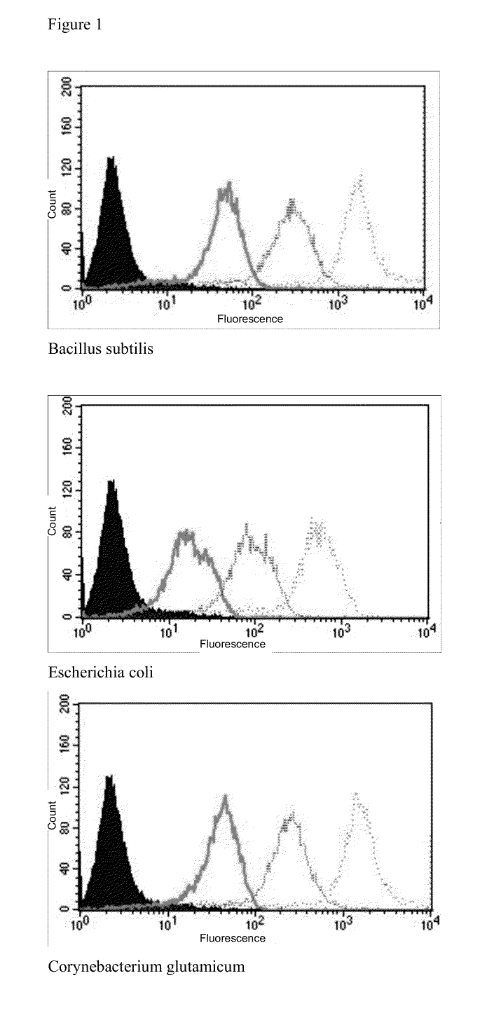

As shown in FIG. 1, the three different microorganisms were bound to the THP1 cells to the same extent at the different microorganism amounts used, and this illustrates that the interaction between the cells of the innate immune system with the microorganisms is a generic interaction and is not specifically restricted to one species or group of microorganisms.

### Example 2

### Binding of Bacillus subtilis to Various Cell Lines

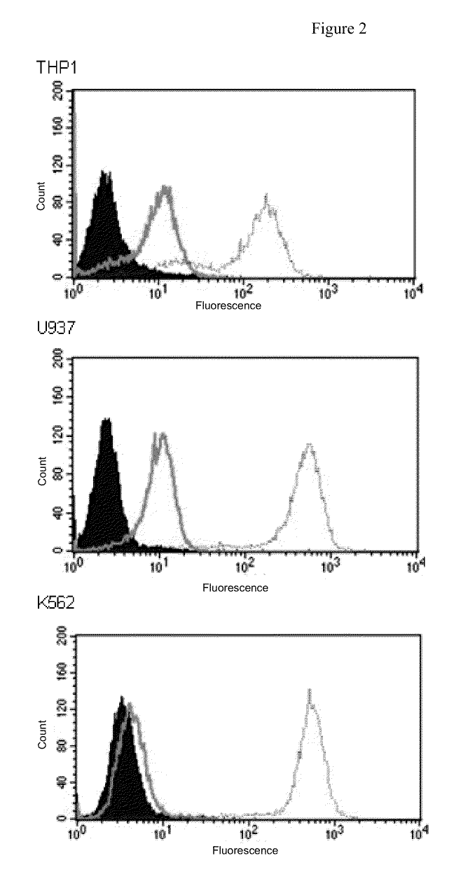

The procedure carried out was as described in example 1, but this time only Bacillus subtilis was used as the microorganism and it was used in amounts of 10⁵ and 10⁷ bacteria. In contrast to example 1, the cells of the innate immune system that were used in example 2 were not only the monocytic cell line THP1 but also the likewise human monocytic cell line U937 and the human B cell line K562. FIG. 2 shows that THP1 and U937 cells bound Bacillus subtilis with about the same efficiency. In contrast, the efficiency was distinctly lower for K562 cells. At an amount of 10⁵ B. subtilis (continuous line), there was hardly a difference in the FACS compared to the control without any B. subtilis (filled area). When, by contrast, 10⁷ B. subtilis were used, about the same efficiency was seen for the cell line K562 as for THP1 and U937. This experiment illustrates that not only cells of the cell line THP1 but also further cell types of the innate immune system are capable of nonspecifically binding microorganisms.

### Example 3

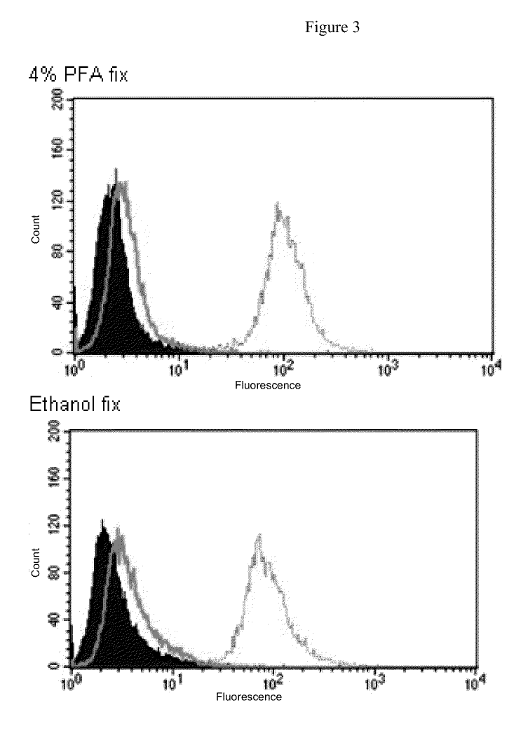

### Binding of Bacillus subtilis to Fixed THP1 Cells

Whereas examples 1 and 2 used THP1 cells which exhibited metabolic activity, example 3 used THP1 cells which had been fixed. Two different methods were used for fixation. In the first fixation procedure, 2×10⁵ THP1 cells were incubated in 4% paraformaldehyde (PFA) for 10 min at room temperature; in the second fixation procedure, incubation was in 100% ethanol for 10 min at room temperature. Whereas paraformaldehyde links the amino residues in proteins, ethanol brings about rapid dehydration of the cells. The rest of the experiments were carried out as described in example 2, using Bacillus subtilis as the microorganism. FIG. 3 shows that microorganisms can be bound not only by cells of the innate immune system exhibiting metabolic activity, but also by fixed cells. Although the detection of 10⁵ microorganisms in this experiment was less efficient than the detection with cells still exhibiting metabolic activity, it is possible even with fixed cells to reliably detect 10⁵ microorganisms in FACS, after they had been accumulated from the starting material using fixed cells of the innate immune system.

### Example 4

### Accumulation of Microorganisms from a Complex Starting Material

The procedure carried out was essentially as described in example 1, but in this experiment 10⁶ and 10⁸ Bacillus subtilis were introduced into the experiments and they were cleaned up from a complex starting material, whole milk with a 1.5% fat content. For this purpose, the microorganisms were added to 25 ml of whole milk and subsequently centrifuged for 5 min in order to pellet the insoluble material including the bacteria. Subsequently, the pellet was collected in 25 ml of phosphate-buffered saline (PBS) and added to 2×10⁵ THP1 cells. Incubation was carried out for 1 or 3 hours at room temperature with rotation.

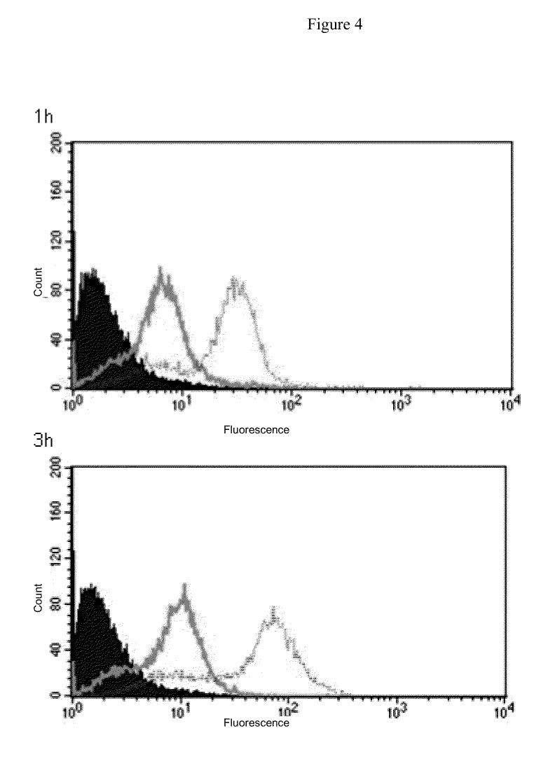

FIG. 4 shows that the bacteria can also be accumulated and detected using the method according to the invention when they are present in a relatively low concentration in a complex starting material such as milk. The results were comparable when the microorganisms had been contacted for 1 or 3 h with the cells of the innate immune system.

### Example 5

### Accumulation of Bacillus subtilis with RT-PCR Detection

10², 10⁴, 10⁶ or 10⁸ Bacillus subtilis were incubated with 2×10⁵ THP1 cells in cell culture medium (RPMI (Invitrogen) containing 10% fetal calf serum (PAA, Pasching, Austria). Unbound microorganisms were removed from the binding complex, either by filtration through a 2 μm PCTE filter (Sterlitech, Kent, USA) or by immunomagnetic separation using a biotinylated CD14 antibody and BiotinBinder magnetic particles (Invitrogen). The genomic DNA from the binding complex was isolated using the QIAamp kit for bacterial gDNA (QIAGEN, Hilden, Germany). The isolated gDNA contained the genetic material of both the THP1 cells and B. subtilis.

5 μl of this eluate were introduced into the subsequent RT-PCR, in which a subregion of the gDNA of B. subtilis was amplified. The total volume of the RT-PCR was 20 μl with the following further components:

10 μl Quantitect Multiplex PCR Mix (QIAGEN)

0.4 μM BacSub-FWD Primer

0.4 μM BacSub-REV Primer

0.2 μM BacSub-Probe

The remaining volume of the reaction mix was made up with water.

The RT-PCR was carried out in 384-well format in an ABI 7900HT running through the following program:

1) 15 min 95° C.

2) 40 cycles of:
15 s 94° C.
30 s 55° C.
30 s 72° C.

The controls used were 10⁶ B. subtilis (100% control) and 2×10⁵ THP1 (0% control).

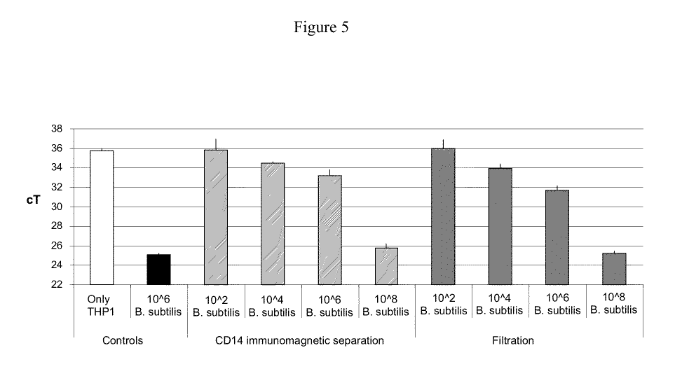

FIG. 5 shows that the microorganisms can also be detected and quantified by means of an RT-PCR after their accumulation with the method according to the invention. As little as 10 000 microorganisms accumulated from the starting material can be detected.

1. A method for nonspecifically accumulating microorganisms from complex starting materials, comprising:
a) contacting the microorganism-containing complex starting material with cells of the innate immune system;
b) forming a binding complex between the microorganisms and cells of the innate immune system; and
c) removing the binding complex from step b) from the complex starting material.

2. The method as claimed in claim 1, characterized in that the cells of the innate immune system originate from mammals.

3. The method as claimed in claim 2, characterized in that the cells of the innate immune system are of human origin.

4. The method as claimed in claim 1, characterized in that the cells are primary cells.

5. The method as claimed in claim 4, characterized in that the primary cells are myeloid cells.

6. The method as claimed in claim 5, characterized in that the myeloid cells are macrophages.

7. The method as claimed in claim 1, characterized in that the cells are immortalized cells of a cell line.

8. The method as claimed in claim 7, characterized in that the immortalized cells of a cell line are derived from the circulatory system.

9. The method as claimed in claim 7, characterized in that the immortalized cells of a cell line are THP1, U937 or K562 or a mixture thereof.

10. The method as claimed in claim 1, characterized in that the cells of the innate immune system exhibit metabolic activity.

11. The method as claimed in claim 1, characterized in that the cells of the innate immune system are immobilized on a solid phase.

12. The method as claimed in claim 1, characterized in that the cells of the innate immune system comprise intracellularly magnetic particles.
